(19) United States
(12) Patent Application Publication
(10) Pub. No.: US 2009/0062756 A1
(43) Pub. Date: Mar. 5, 2009

(54) Signaling Device For Disposable Products

(76) Inventors: Andrew Mark Long, Appleton, WI (US); Donald J. Schmidt, Neenah, WI (US); Thomas Michael Ales, Neenah, WI (US); Val V. Finch, Neenah, WI (US); Keith William Magic, Clarkston, MI (US)

Correspondence Address: DORITY & MANNING, P.A., POST OFFICE BOX 1449, GREENVILLE, SC, 29602-1449, US

(21) Appl. No.: 11/848,714

(22) Filed: Aug. 31, 2007

(51) Int. Cl. A61F 13/15

(52) U.S. Cl. 604/361

(57) ABSTRACT

A signaling device for garments is disclosed. The signaling devices, for instance, may be attached to an absorbent article containing a wetness sensing system. In particular, the signaling device may be configured to emit an audible and/or visible signal when a conductive fluid is sensed in the absorbent article. In accordance with the present disclosure, the signaling device is designed based upon anthropomorphic data so that the signaling device can fit within the small of the back of a wearer while minimizing discomfort. The signaling device can also be made from various materials that provide some protection to the internal components of the signaling device while also minimizing discomfort.

### BACKGROUND

Absorbent articles such as diapers, training pants, incontinence products, feminine hygiene products, swim undergarments, and the like conventionally include a liquid permeable body-side liner, a liquid impermeable outer cover, and an absorbent core. The absorbent core is typically located in between the outer cover and the liner for taking in and retaining liquids (e.g., urine) exuded by the wearer.

The absorbent core can be made of, for instance, superabsorbent particles. Many absorbent articles, especially those sold under the tradename HUGGIES™ by the Kimberly-Clark Corporation, are so efficient at absorbing liquids that it is sometimes difficult to tell whether or not the absorbent article has been insulted with a body fluid.

Accordingly, various types of moisture or wetness indicators have been suggested for use in absorbent articles. The wetness indicators may include alarm devices that are designed to assist parents or attendants identify a wet diaper condition early on. The devices can produce, for instance, an audible signal and/or a visual signal.

The alarm devices are typically configured to be attached around the waist of the absorbent article when the article is being worn. When attached to an absorbent article, however, the alarm device may provide some discomfort to the wearer. As such, a need currently exists for an alarm device for attachment to an absorbent article that minimizes discomfort.

### SUMMARY

The present disclosure is generally directed to a signaling device that is particularly well suited for use in conjunction with absorbent articles. The signaling device, for instance, may be connected to a wetness sensor in the absorbent article and can be configured to emit a signal, such as an audible or visual signal, for indicating to a user that a body fluid is present in the absorbent article. For example, in one embodiment, the absorbent article comprises a diaper and the wetness sensing device in conjunction with the signaling device is configured to indicate the presence of urine or a bowel movement. In other absorbent articles, however, the wetness sensing device and the signaling device may be configured to indicate the presence of yeast or metabolites.

More particularly, the present disclosure is directed to a signaling device as described above that has been anthropomorphically designed so as to minimize discomfort when worn in conjunction with an absorbent article. For example, in one embodiment, the signaling device may be configured to attach to the back waistband of an absorbent article, such as a diaper or training pant, and have a shape such that the signaling device fits within and conforms to the small of the back of the wearer. The signaling device can be designed, for instance, so as to have particular dimensions and can be made from particular materials that have been found to create as little pressure as possible against the back of a wearer.

For example, in one embodiment, the signaling device includes an attachment mechanism for attaching the signaling device to a garment, such as an absorbent article. The signaling device can further include a housing containing internal components that are designed to emit any suitable signal, such as an audible and/or visible signal when activated.

The housing of the signaling device has a first side configured to contact a user when attached to a garment and a second and opposite side. The first side and the second side define an X length and a Y length. The housing further has a thickness defining a Z length. In accordance with the present disclosure, the X length and the Y length can be from about 30 mm to about 80 mm, such as from about 30 mm to about 50 mm, such as from about 35 mm to about 55 mm.

The Z length, on the other hand, is generally less than about 15 mm. For instance, the Z length can be from about 5 mm to about 10 mm in one embodiment.

The first side of the housing can include at least two rounded corners in certain embodiments. The corners are rounded such that the radii of curvature (which can be the same as the radii) at the two corners between an edge in the Z direction and an edge in either the X direction or the Y direction is greater than about 1.5 mm⁻¹. For instance, the radii of curvature can be from about 1.5 mm⁻¹ to about 15 mm⁻¹, such as from about 1.5 mm⁻¹ to about 5 mm⁻¹.

The first side of the housing can have various shapes in accordance with the present disclosure. In one embodiment, for instance, the first side of the housing can generally having a rectangular shape. In other embodiments, the first side may only include two corners. For instance, the first side of the housing may have an oval-like shape that extends from the two corners.

In an alternative embodiment, the first side of the housing may only include three corners. In this embodiment, for instance, the first side may have a triangular shape. In one particular embodiment, the first side might have a shield-like shape. In still another embodiment, the first side of the housing may have three flat sides and one arcuate-shaped side.

The housing of the signaling device can be made from various materials. In one embodiment, for instance, the housing can be made from a polymer, such as any suitable thermoplastic or elastomeric polymer. In one embodiment, the first side of the housing is made from a material such that the first side has a Shore A hardness of from about 25 A to about 75 A. In addition, the first side can be made from a material having a modulus of less than about 10 GPa.

In one embodiment, the housing and the signaling device can be made from a first section and a second section. The first section and the second section can be, for instance, assembled together in any suitable way. In one particular embodiment, for instance, the first section may be connected to the second section about a hinge. The first section may define the first side and can be made from a material having the properties described above. The second section, on the other hand, may define the second side and can be made from a harder material. For instance, the second section can be made from a material having a Shore A hardness of from about 60 A to about 110 A. In addition, the material used to form the second section can have a modulus of greater than about 12 GPa.

The signaling device of the present disclosure is particularly well suited for use with wetness sensing systems in absorbent articles. In this regard, the present disclosure is also directed to an absorbent article. The absorbent article can have an absorbent core positioned between a bodyside liner and an outer cover. The absorbent article may define a waist opening having a front and a back and two opposing leg openings when worn. The absorbent article may further comprise a wetness sensing circuit. The wetness sensing circuit may be configured to attach to the signaling device along the back of the waist opening so that the signaling device fits within the small of the back of the wearer.

Of particular advantage, when a signaling device made in accordance with the present disclosure is attached to an absorbent article as described above, the signaling device applies only relatively low amounts of pressure on the person's back. For instance, the signaling device may exert an average pressure on the back of less than about 3 psi, such as less than about 2.5 psi, such as less than about 2 psi even when the wearer is lying on his or her back. Similarly, the perimeter pressure of the signaling device can also be less than 3 psi, such as less than about 2.5 psi.

The absorbent article used with the signaling device can vary. The absorbent articles that may be made in accordance with the present disclosure include, for instance, diapers, training pants, swim pants, adult incontinence products, feminine hygiene products, and the like.

Other features and aspects of the present invention are discussed in greater detail below.

### BRIEF DESCRIPTION OF THE DRAWINGS

A full and enabling disclosure of the present invention, including the best mode thereof to one skilled in the art, is set forth more particularly in the remainder of the specification, including reference to the accompanying figures, in which:

FIG. 1 is a rear perspective view of one embodiment of an absorbent article made in accordance with the present invention;

FIG. 2 is a front perspective view of the absorbent article illustrated in FIG. 1;

FIG. 3 is a plan view of the absorbent article shown in FIG. 1 with the article in an unfastened, unfolded and laid flat condition showing the surface of the article that faces away from the wearer;

FIG. 4 is a plan view similar to FIG. 3 showing the surface of the absorbent article that faces the wearer when worn and with portions cut away to show underlying features;

FIG. 5 is a perspective view of the embodiment shown in FIG. 1 further including one embodiment of a signaling device;

FIG. 6 is a plan view of one embodiment of a signaling device made in accordance with the present disclosure;

FIG. 7 is a side view of the signaling device illustrated in FIG. 6;

FIG. 8 is a plan view of an alternative embodiment of a signaling device made in accordance with the present disclosure;

FIG. 9 is a plan view of still another embodiment of a signaling device made in accordance with the present disclosure;

FIG. 10 is a plan view of yet another embodiment of a signaling device made in accordance with the present disclosure;

FIG. 11 are perspective views of the shapes tested in the example below; and

FIGS. 12 and 13 are graphical representations of the results obtained in the example below.

Repeat use of reference characters in the present specification and drawings is intended to represent the same or analogous features or elements of the present invention.

### DETAILED DESCRIPTION

It is to be understood by one of ordinary skill in the art that the present discussion is a description of exemplary embodiments only, and is not intended as limiting the broader aspects of the present disclosure.

The present disclosure is generally directed to signaling devices for absorbent articles. The signaling devices are configured to communicate with a wetness sensing device contained within the absorbent article. When a conductive substance is detected within the absorbent article, the wetness sensing device is activated causing the signaling device to emit any suitable signal, such as an audible and/or visual signal. The absorbent article may be, for instance, a diaper, a training pant, an incontinence product, a feminine hygiene product, a medical garment, a bandage, and the like. Absorbent articles made according to the present disclosure may include, for instance, an open circuit that becomes closed when a conductive fluid, such as a body fluid, is sensed in between a pair of conductive leads. Generally, the absorbent articles containing the open circuit are disposable meaning that they are designed to be discarded after a limited use rather than being laundered or otherwise reused.

The open circuit contained within the absorbent articles of the present disclosure is configured to be attached to the signaling device. The signaling device can provide power to the open circuit while also including some type of signal that indicates to the user the presence of a body fluid. Although the absorbent article itself is disposable, the signaling device may be reusable from article to article.

In accordance with the present disclosure, the signaling device is anthropomorphically designed so as to minimize discomfort to a user when attached and worn with a garment, such as an absorbent article as described above. For instance, the signaling device can include a housing that has a shape and can be made from materials that lessen any impingement on the skin of the wearer.

In one particular embodiment, the signaling device is designed to be attached to the rear waistband of an absorbent article such that the device lays adjacent to a user's back. In this regard, in one embodiment, the signaling device can have a shape designed to fit within the small of the back of the wearer. The small of the back is generally defined as the area of the back where the lumbar vertebrae are located. The lumbar vertebrae forms what is referred to as the lumbar curve which creates a concave portion on the body between one's buttocks and the middle of the back.

The signaling device is typically connected to an open circuit that is configured to indicate the presence of a body fluid contained within an absorbent article. The particular targeted body fluid may vary depending upon the particular type of absorbent article and the desired application. For instance, in one embodiment, the absorbent article comprises a diaper, a training pant, or the like and the signaling device is configured to indicate the presence of urine. Alternatively, the signaling device may be configured to indicate the presence of a metabolite that would indicate the presence of a diaper rash. For adult incontinence products and feminine hygiene products, on the other hand, the signaling device may be configured to indicate the presence of a yeast or of a particular constituent in urine, such as a polysaccharide.

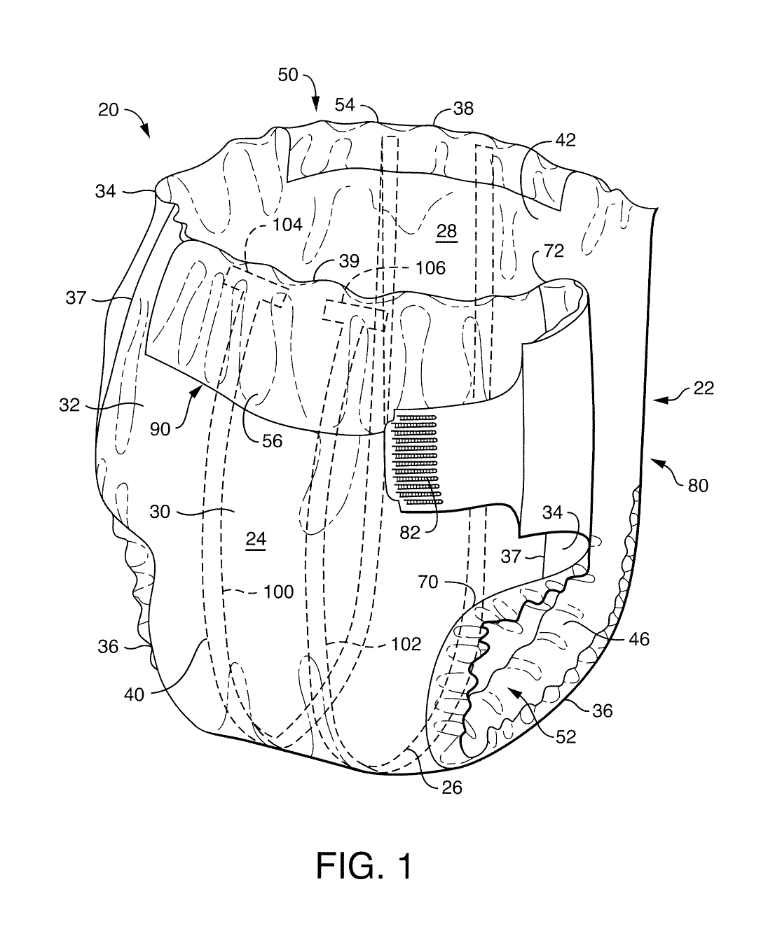

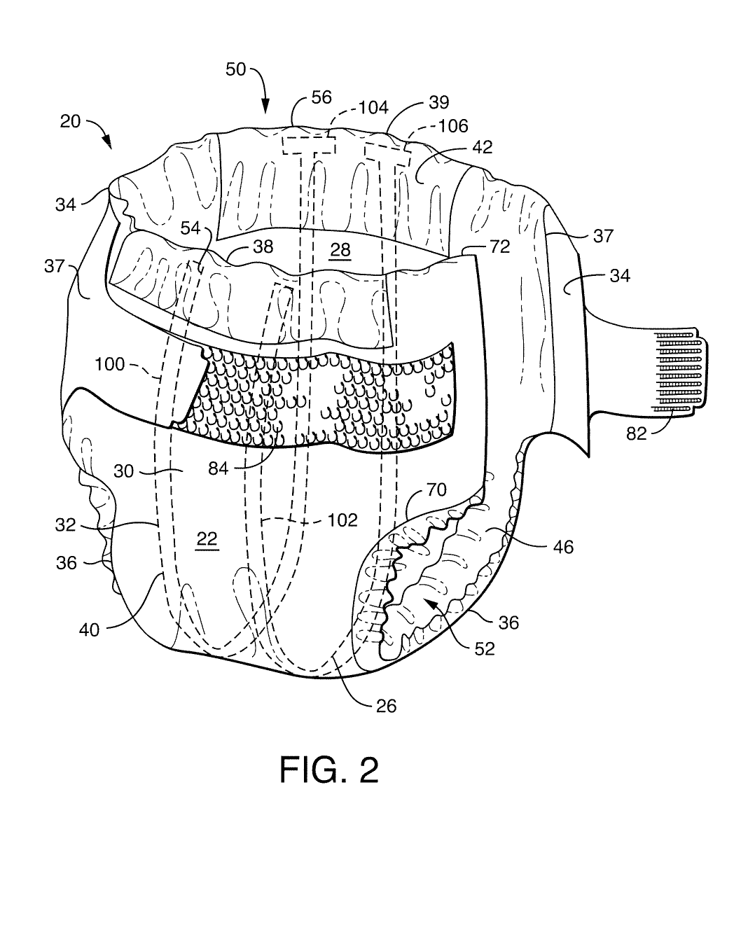

Referring to FIGS. 1 and 2, for exemplary purposes, an absorbent article 20 that may be made in accordance with the present disclosure is shown. The absorbent article 20 may or may not be disposable. It is understood that the present disclosure is suitable for use with various other absorbent articles intended for personal wear, including but not limited to diapers, training pants, swim pants, feminine hygiene products, incontinence products, medical garments, surgical pads and bandages, other personal care or health care garments, and the like without departing from the scope of the present invention.

By way of illustration only, various materials and methods for constructing absorbent articles such as the diaper 20 of the various aspects of the present invention are disclosed in PCT Patent Application WO 00/37009 published Jun. 29, 2000 by A. Fletcher et al; U.S. Pat. No. 4,940,464 issued Jul. 10, 1990 to Van Gompel et al.; U.S. Pat. No. 5,766,389 issued Jun. 16, 1998 to Brandon et al., and U.S. Pat. No. 6,645,190 issued Nov. 11, 2003 to Olson et al. which are incorporated herein by reference to the extent they are consistent (i.e., not in conflict) herewith.

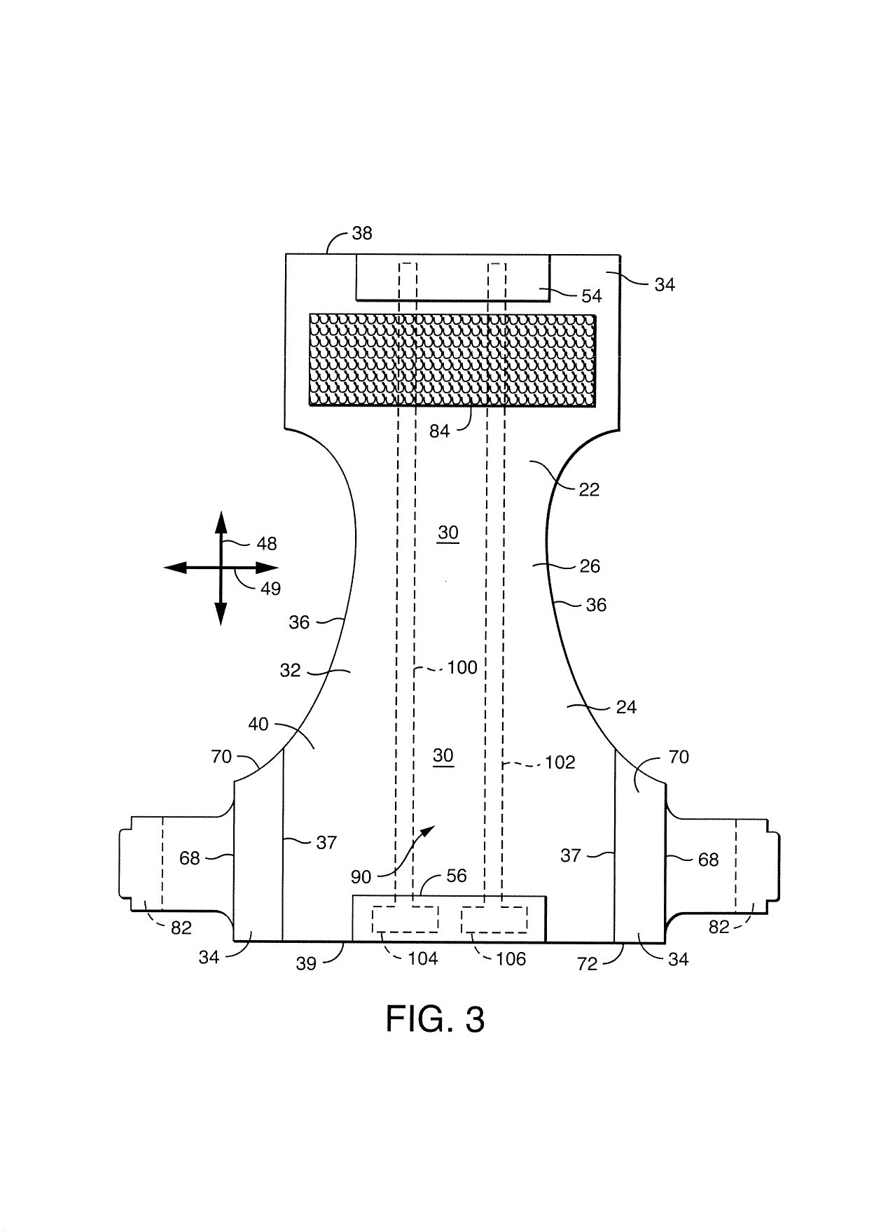

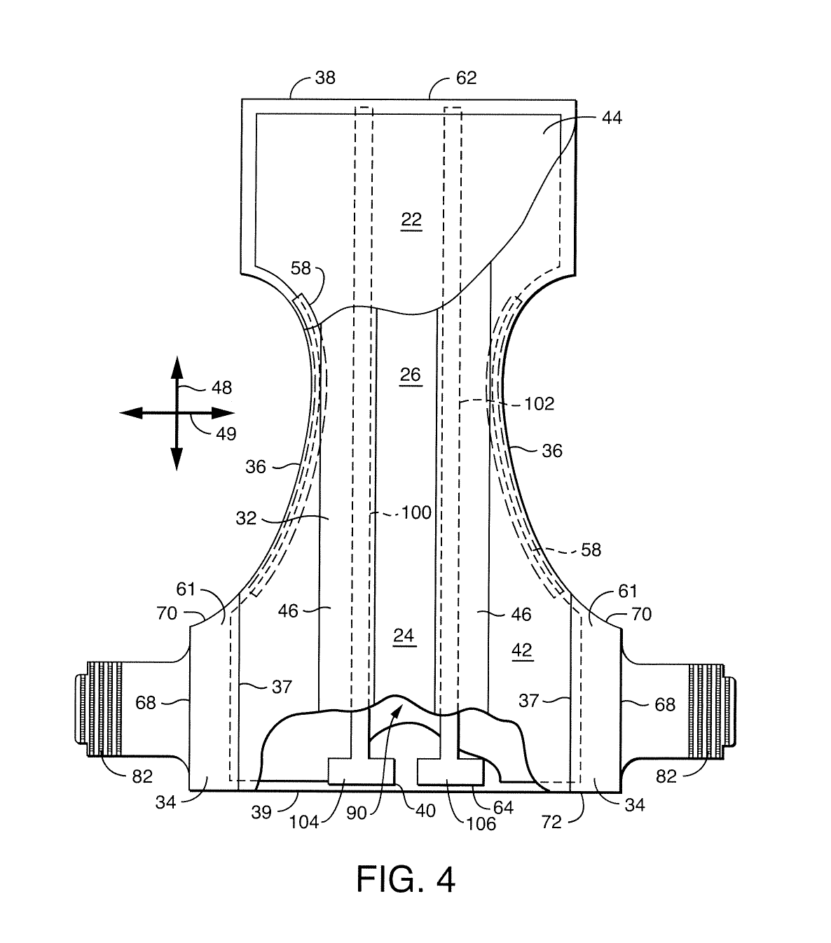

A diaper 20 is representatively illustrated in FIG. 1 in a partially fastened condition. The diaper 20 shown in FIGS. 1 and 2 is also represented in FIGS. 3 and 4 in an opened and unfolded state. Specifically, FIG. 3 is a plan view illustrating the exterior side of the diaper 20, while FIG. 4 illustrates the interior side of the diaper 20. As shown in FIGS. 3 and 4, the diaper 20 defines a longitudinal direction 48 that extends from the front of the article when worn to the back of the article. Opposite to the longitudinal direction 48 is a lateral direction 49.

The diaper 20 defines a pair of longitudinal end regions, otherwise referred to herein as a front region 22 and a back region 24, and a center region, otherwise referred to herein as a crotch region 26, extending longitudinally between and interconnecting the front and back regions 22, 24. The diaper 20 also defines an inner surface 28 adapted in use (e.g., positioned relative to the other components of the article 20) to be disposed toward the wearer, and an outer surface 30 opposite the inner surface. The front and back regions 22, 24 are those portions of the diaper 20, which when worn, wholly or partially cover or encircle the waist or mid-lower torso of the wearer. The crotch region 26 generally is that portion of the diaper 20 which, when worn, is positioned between the legs of the wearer and covers the lower torso and crotch of the wearer. The absorbent article 20 has a pair of laterally opposite side edges 36 and a pair of longitudinally opposite waist edges, respectively designated front waist edge 38 and back waist edge 39.

The illustrated diaper 20 includes a chassis 32 that, in this embodiment, encompasses the front region 22, the back region 24, and the crotch region 26. Referring to FIGS. 1-4, the chassis 32 includes an outer cover 40 and a bodyside liner 42 (FIGS. 1 and 4) that may be joined to the outer cover 40 in a superimposed relation therewith by adhesives, ultrasonic bonds, thermal bonds or other conventional techniques. Referring to FIG. 4, the liner 42 may suitably be joined to the outer cover 40 along the perimeter of the chassis 32 to form a front waist seam 62 and a back waist seam 64. As shown in FIG. 4, the liner 42 may suitably be joined to the outer cover 40 to form a pair of side seams 61 in the front region 22 and the back region 24. The liner 42 can be generally adapted, i.e., positioned relative to the other components of the article 20, to be disposed toward the wearer's skin during wear of the absorbent article. The chassis 32 may further include an absorbent structure 44 particularly shown in FIG. 4 disposed between the outer cover 40 and the bodyside liner 42 for absorbing liquid body exudates exuded by the wearer, and may further include a pair of containment flaps 46 secured to the bodyside liner 42 for inhibiting the lateral flow of body exudates.

The elasticized containment flaps 46 as shown in FIG. 4 define a partially unattached edge which assumes an upright configuration in at least the crotch region 26 of the diaper 20 to form a seal against the wearer's body. The containment flaps 46 can extend longitudinally along the entire length of the chassis 32 or may extend only partially along the length of the chassis. Suitable constructions and arrangements for the containment flaps 46 are generally well known to those skilled in the art and are described in U.S. Pat. No. 4,704,116 issued Nov. 3, 1987 to Enloe, which is incorporated herein by reference.

To further enhance containment and/or absorption of body exudates, the diaper 20 may also suitably include leg elastic members 58 (FIG. 4), as are known to those skilled in the art. The leg elastic members 58 can be operatively joined to the outer cover 40 and/or the bodyside liner 42 and positioned in the crotch region 26 of the absorbent article 20.

The leg elastic members 58 can be formed of any suitable elastic material. As is well known to those skilled in the art, suitable elastic materials include sheets, strands or ribbons of natural rubber, synthetic rubber, or thermoplastic elastomeric polymers. The elastic materials can be stretched and adhered to a substrate, adhered to a gathered substrate, or adhered to a substrate and then elasticized or shrunk, for example with the application of heat, such that elastic retractive forces are imparted to the substrate. In one particular aspect, for example, the leg elastic members 58 may include a plurality of dry-spun coalesced multifilament spandex elastomeric threads sold under the trade name LYCRA and available from Invista, Wilmington, Del., U.S.A.

In some embodiments, the absorbent article 20 may further include a surge management layer (not shown) which may be optionally located adjacent the absorbent structure 44 and attached to various components in the article 20 such as the absorbent structure 44 or the bodyside liner 42 by methods known in the art, such as by using an adhesive. A surge management layer helps to decelerate and diffuse surges or gushes of liquid that may be rapidly introduced into the absorbent structure of the article. Desirably, the surge management layer can rapidly accept and temporarily hold the liquid prior to releasing the liquid into the storage or retention portions of the absorbent structure. Examples of suitable surge management layers are described in U.S. Pat. No. 5,486,166; and U.S. Pat. No. 5,490,846. Other suitable surge management materials are described in U.S. Pat. No. 5,820,973. The entire disclosures of these patents are hereby incorporated by reference herein to the extent they are consistent (i.e., not in conflict) herewith.

As shown in FIGS. 1-4, the absorbent article 20 further includes a pair of opposing elastic side panels 34 that are attached to the back region of the chassis 32. As shown particularly in FIGS. 1 and 2, the side panels 34 may be stretched around the waist and/or hips of a wearer in order to secure the garment in place. As shown in FIGS. 3 and 4, the elastic side panels are attached to the chassis along a pair of opposing longitudinal edges 37. The side panels 34 may be attached or bonded to the chassis 32 using any suitable bonding technique. For instance, the side panels 34 may be joined to the chassis by adhesives, ultrasonic bonds, thermal bonds, or other conventional techniques.

In an alternative embodiment, the elastic side panels may also be integrally formed with the chassis 32. For instance, the side panels 34 may comprise an extension of the bodyside liner 42, of the outer cover 40, or of both the bodyside liner 42 and the outer cover 40.

In the embodiments shown in the figures, the side panels 34 are connected to the back region of the absorbent article 20 and extend over the front region of the article when securing the article in place on a user. It should be understood, however, that the side panels 34 may alternatively be connected to the front region of the article 20 and extend over the back region when the article is donned.

With the absorbent article 20 in the fastened position as partially illustrated in FIGS. 1 and 2, the elastic side panels 34 may be connected by a fastening system 80 to define a 3-dimensional diaper configuration having a waist opening 50 and a pair of leg openings 52. The waist opening 50 of the article 20 is defined by the waist edges 38 and 39 which encircle the waist of the wearer.

In the embodiments shown in the figures, the side panels are releasably attachable to the front region 22 of the article 20 by the fastening system. It should be understood, however, that in other embodiments the side panels may be permanently joined to the chassis 32 at each end. The side panels may be permanently bonded together, for instance, when forming a training pant or absorbent swimwear.

The elastic side panels 34 each have a longitudinal outer edge 68, a leg end edge 70 disposed toward the longitudinal center of the diaper 20, and waist end edges 72 disposed toward a longitudinal end of the absorbent article. The leg end edges 70 of the absorbent article 20 may be suitably curved and/or angled relative to the lateral direction 49 to provide a better fit around the wearer's legs. However, it is understood that only one of the leg end edges 70 may be curved or angled, such as the leg end edge of the back region 24, or alternatively, neither of the leg end edges may be curved or angled, without departing from the scope of the present invention. As shown in FIG. 4, the outer edges 68 are generally parallel to the longitudinal direction 48 while the waist end edges 72 are generally parallel to the transverse axis 49. It should be understood, however, that in other embodiments the outer edges 68 and/or the waist edges 72 may be slanted or curved as desired. Ultimately, the side panels 34 are generally aligned with a waist region 90 of the chassis.

The fastening system 80 may include laterally opposite first fastening components 82 adapted for refastenable engagement to corresponding second fastening components 84. In the embodiment shown in the figures, the first fastening component 82 is located on the elastic side panels 34, while the second fastening component 84 is located on the front region 22 of the chassis 32. In one aspect, a front or outer surface of each of the fastening components 82, 84 includes a plurality of engaging elements. The engaging elements of the first fastening components 82 are adapted to repeatedly engage and disengage corresponding engaging elements of the second fastening components 84 to releasably secure the article 20 in its three-dimensional configuration.

The fastening components 82, 84 may be any refastenable fasteners suitable for absorbent articles, such as adhesive fasteners, cohesive fasteners, mechanical fasteners, or the like. In particular aspects the fastening components include mechanical fastening elements for improved performance. Suitable mechanical fastening elements can be provided by interlocking geometric shaped materials, such as hooks, loops, bulbs, mushrooms, arrowheads, balls on stems, male and female mating components, buckles, snaps, or the like.

In the illustrated aspect, the first fastening components 82 include hook fasteners and the second fastening components 84 include complementary loop fasteners. Alternatively, the first fastening components 82 may include loop fasteners and the second fastening components 84 may be complementary hook fasteners. In another aspect, the fastening components 82, 84 can be interlocking similar surface fasteners, or adhesive and cohesive fastening elements such as an adhesive fastener and an adhesive-receptive landing zone or material; or the like. One skilled in the art will recognize that the shape, density and polymer composition of the hooks and loops may be selected to obtain the desired level of engagement between the fastening components 82, 84. Suitable fastening systems are also disclosed in the previously incorporated PCT Patent Application WO 00/37009 published Jun. 29, 2000 by A. Fletcher et al. and the previously incorporated U.S. Pat. No. 6,645,190 issued Nov. 11, 2003 to Olson et al.

In the embodiment shown in the figures, the fastening components 82 are attached to the side panels 34 along the edges 68. In this embodiment, the fastening components 82 are not elastic or extendable. In other embodiments, however, the fastening components may be integral with the side panels 34. For example, the fastening components may be directly attached to the side panels 34 on a surface thereof.

In addition to possibly having elastic side panels, the absorbent article 20 may include various waist elastic members for providing elasticity around the waist opening. For example, as shown in the figures, the absorbent article 20 can include a front waist elastic member 54 and/or a back waist elastic member 56.

As described above, the present disclosure is particularly directed to incorporating a body fluid indicating system, such as a wetness sensing device into the absorbent article 20. In this regard, as shown in FIGS. 1-4, the absorbent article 20 includes a first conductive element 100 spaced from a second conductive element 102. In this embodiment, the conductive elements extend from the front region 22 of the absorbent article to the back region 24 without intersecting. The conductive elements 100 and 102 can comprise any suitable conductive material, such as a conductive thread or a conductive foil. The first conductive element 100 does not intersect the second conductive element 102 in order to form an open circuit that may be closed, for instance, when a conductive fluid is positioned in between the conductive elements. In other embodiments, however, the first conductive element 100 and the second conductive element 102 may be connected to a sensor within the chassis. The sensor may be used to sense changes in temperature or may be used to sense the presence of a particular substance, such as a metabolite.

In the embodiment shown in FIG. 1, the conductive elements 100 and 102 extend the entire length of the absorbent article 20. It should be understood, however, that in other embodiments the conductive elements may extend only to the crotch region 26 or may extend to any particular place in the absorbent article where a body fluid is intended to be sensed.

The conductive elements 100 and 102 may be incorporated into the chassis 32 at any suitable location as long as the conductive elements are positioned so as to contact a body fluid that is absorbed by the absorbent article 20. In this regard, the conductive elements 100 and 102 generally lie inside the outer cover 40. In fact, in one embodiment, the conductive elements 100 and 102 may be attached or laminated to the inside surface of the outer cover 40 that faces the absorbent structure 44. Alternatively, however, the conductive elements 100 and 102 may be positioned on the absorbent structure 44 or positioned on the liner 42.

In order for the conductive elements 100 and 102 to be easily connected to a signaling device, the first conductive element 100 is attached to a first conductive pad member 104, while the second conductive element 102 is connected to a second conductive pad member 106. The pad members 104 and 106 are provided for making a reliable connection between the open circuit formed by the conductive elements to a signaling device that is intended to be installed on the chassis by the consumer.

The position of the conductive pad members 104 and 106 on the absorbent article 20 can vary depending upon where it is desired to mount the signaling device. For instance, in the figures, the conductive pad members 104 and 106 are positioned in the back region 24 along the waist opening of the article. In still other embodiments, it should be understood that the pad members may be located in front, along the side of the article or towards the crotch region of the article.

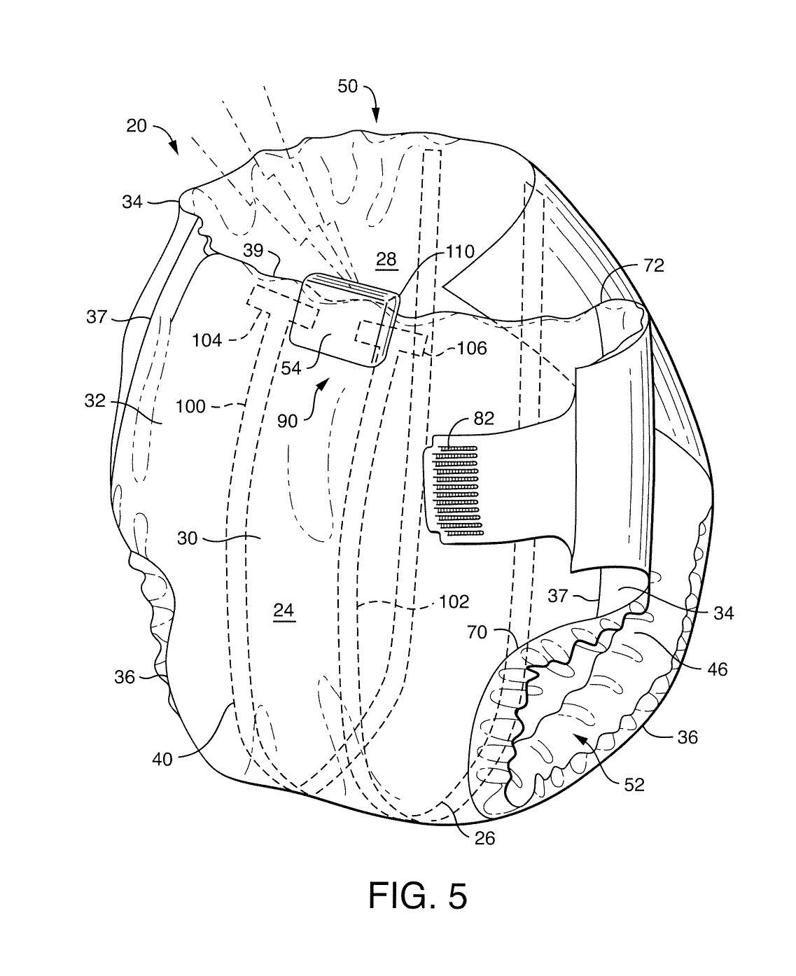

Referring to FIG. 5, for exemplary purposes, a signaling device 110 is shown attached to the conductive pad members 104 and 106. The signaling device 110 includes a pair of opposing terminals that are electrically connected to the corresponding conductive pad members. When a body fluid is present in the absorbent article 20, the open circuit formed by the conductive elements 100 and 102 is closed which, in turn, activates the signaling device 110.

The signaling device 110 can emit an audible and/or visual signal in order to indicate to the user that the circuit has been closed.

As described above, the present disclosure is particularly directed to designing the signaling device so that the signaling device minimizes any discomfort when worn. In addition to being comfortable when attached to an absorbent article, however, the signaling device must also be durable. Based upon anthropomorphic modeling, signaling devices made in accordance with the present disclosure are produced with dimensions that fall within a set of desirable ranges. In addition, the signaling device can also include radii on the corners of the signaling device that are constrained by a set of desirable ranges to ensure that the corners do not impinge, scratch or poke the body's surface.

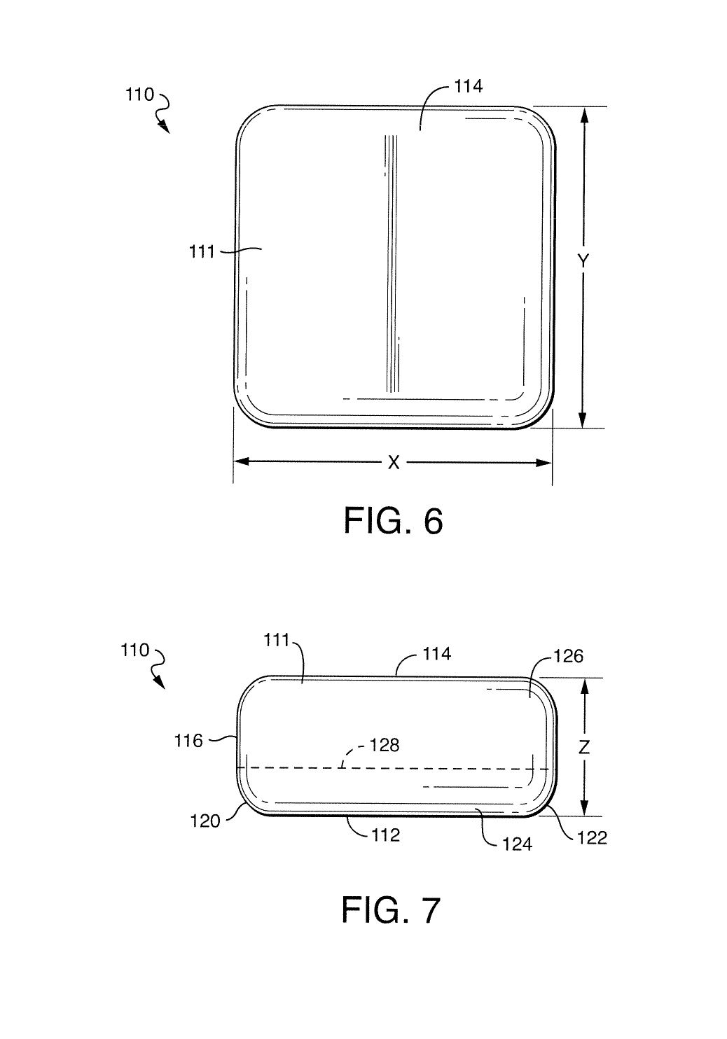

Referring to FIGS. 6 and 7, one embodiment of a signaling device 110 that may be made in accordance with the present disclosure is shown. The signaling device 110 can include a housing 111 that contains the internal components. The internal components, for instance, may include a battery and may be configured to generate an audible and/or a visible signal. As illustrated, the signaling device in this embodiment generally has a rectangular shape, such as a square shape, with rounded corners.

Not shown, the signaling device can also include an attachment mechanism for attaching the device to an absorbent article. In general, any suitable attachment mechanism may be used. For instance, the attachment mechanism may comprise a clip, one or more snaps, a hook or loop fastener, or the like. In one embodiment, the attachment mechanism may not only attach the signaling device to an absorbent article, but also may make an electrical connection between the signaling device and a wetness sensing system contained in the article. Suitable attachment mechanisms that may be used include those disclosed in U.S. patent application Ser. No. 11/405,263, which is incorporated herein by reference.

As shown in FIGS. 6 and 7, the signaling device 110 includes a first surface 112 that is configured to be placed adjacent to a user, such as the user's back. The signaling device 110 further includes a second and opposite surface 114. As shown in FIG. 6, the first and second surfaces 112 and 114 generally extend in an X-Y plane and have an X length and a Y length. For most applications, at least either the X length or the Y length of the signaling device 110 should be greater than about 30 mm. For example, both the X length and the Y length of the signaling device can be greater than about 30 mm. As the X length or Y length increases, however, the signaling device 110 has greater opportunity to impinge on the skin of a user due to the curvature of one's back. Thus, in one embodiment, both the X length and the Y length are no greater than about 80 mm.

The actual dimensions in the X direction and the Y direction, however, depend upon numerous factors including the age, weight and size of the user, the types of materials used to construct the signaling device, and the thickness of the signaling device. In one embodiment, for instance, the X direction and the Y direction can be from about 30 mm to about 45 mm. In an alternative embodiment, however, a more comfortable device may be constructed if the X distance and the Y distance are from about 35 mm to about 55 mm. In one embodiment, the signaling device can have dimensions that satisfy the requirements of the Child Safety Protection Act. For example, in one embodiment, the signaling device can have an actual diameter or a constructive diameter of greater than about 1.25 inches.

As shown in FIG. 7, the signaling device 110 also has a thickness 116 that can extend generally in the Z direction. The thickness 116, for instance, can have a Z length that is generally less than about 15 mm. Thicknesses greater than about 15 mm, for instance, may create a signaling device that is excessively bulky in some applications. In general, the Z length can be from about 5 mm to about 15 mm, such as from about 5 mm to about 10 mm. In certain embodiments, the thickness may even be less. Depending upon the type of material used to construct the housing 111 of the signaling device 110, however, reduced thicknesses may create edges and/or corners that can impinge upon the skin of the user.

In the embodiment of the signaling device 110 illustrated in FIGS. 6 and 7, the first surface 112 and the second surface 114 include four rounded corners. The corners are rounded not only in the X-Y plane as particularly shown in FIG. 6, but also in the Z-Y plane and in the Z-X plane as shown in FIG. 7. For instance, referring to FIG. 7, the first side 112 of the signaling device 110 includes four corners that are intended to be in contact with the back of a wearer, such as corners 120 and 122. In accordance with the present disclosure, the radii and/or the radii of curvature of the four corners of the first side 112 between an edge in the Z direction and an edge in the X direction and in the Y direction should be within desired ranges so as to minimize impingement on the skin or other discomfort. For instance, in one embodiment, the radii of curvature of corners 120 and 122 should be greater than about 1.5 mm⁻¹, such as from about 1.5 mm⁻¹ to about 15 mm⁻¹. More particularly, the radii of curvature of the corners contacting the wearer in either the X-Z plane or in the Y-Z plane can be from about 1.5 mm⁻¹ to about 5 mm⁻¹. The actual radius of curvature of the corners, however, may depend upon various factors, including the type of material used to form the housing 111.

In addition to forming rounded corners in the X-Z plane or in the Y-Z plane at a location contiguous with the first surface 112, it should be understood that the other various corners can also be rounded in order to improve comfort and/or to improve aesthetics. For instance, as shown in FIG. 6, the corners in the X-Y plane that make up the second surface 114 or the first surface 112 can also be rounded and can have a radii as described above.

The housing 111 as shown in FIGS. 6 and 7 can be made from any suitable material. In one embodiment, for instance, the housing 111 can be made from a polymer, such as a thermoplastic polymer or an elastomeric polymer. In one embodiment of the present disclosure, the housing 111 can be made from materials that further decrease or minimize discomfort when the signaling device 110 is placed adjacent to a person's body. For example, in addition to the dimensions as described above, forming the housing 111 at least in part from soft materials can further improve the risk of discomfort to the user. As described above, however, the housing 111 should also be made from durable materials that will be able to withstand any impacts that the signaling device may be subjected to during normal wear. For instance, the housing 111 should be somewhat shock/crush resistant to protect the integrity of the internal components.

Thus, another aspect of the present disclosure relates to the materials used to construct the housing 111. In accordance with the present disclosure, for instance, the housing 111 can be constructed from one or more materials having a hardness within a desired range. For example, durometer is one of several ways to indicate the hardness of a material. Durometer is defined as the resistance of the material to permanent indentation. Durometer is typically used as a measure of hardness in polymers, elastomers and rubbers. There are several scales of durometer used for materials with different properties. For example, durometer can be measured according to Shore A hardness or according to Shore D hardness. The A scale is typically used for softer polymers, while the D scale is typically used for harder polymers.

In one embodiment of the present disclosure, the present inventors have discovered that a desirable mix of properties is obtained when the first surface 112 of the housing 111 is made from a material having a Shore A hardness of from about 25 A to about 70 A. For example, the first surface 112 of the housing 111 can be made from various different thermoplastic and elastomeric polymers including silicone polymers, polyurethanes, polystyrenes, polyesters, polycarbonates, polyolefins, elastomeric polyolefin materials, such as elastomeric polyethylenes and polypropylenes including copolymers thereof, styrenic block copolymers including S-I-S polymers, S-EB-S polymers, SBR polymers, nitrile polymers, natural latex polymers, plasticized polyvinyl chloride polymers, and the like.

In addition to having a hardness within the above range, the material used to form the exterior of the first surface 112 can also have a Young's modulus (E) within a desired range. For instance, the modulus of the material can be less than about 10 GPa, such as less than about 6 GPa.

In one embodiment, the entire housing 111 can be made from a material as described above. Alternatively, the housing 111 can have a multi-layer structure in which the housing includes a core made from a harder material and an outer covering made from a material as described above.

In still another embodiment, the housing 111 can be made from multiple parts wherein each part is made from a different material. For instance, in one embodiment, as shown in FIG. 7, the housing 111 can include a first section 124 attached to a second section 126. The first section 124 can be attached to the second section 126 in any suitable manner. In one particular embodiment, for instance, the first section 124 can be connected to the second section 126 about a hinge. In this embodiment, for instance, the signaling device may include a hinge line 128 as shown in FIG. 7. In this embodiment, the first section 124 can be made from a more flexible material than the second section 126. Flexibility of the housing 111 along the first surface 112 can provide comfort to the user. The second section 126, on the other hand, can be made from a harder material that can serve as a protective shell for the interior components of the signaling device.

In one embodiment, for instance, the first section 124 can be made from a material having a hardness and modulus as described above. The second section 126, on the other hand, can be made from a material having a Shore A hardness of from about 60 A to about 110 A or alternatively from a material having a Shore D hardness of from about 50 D to about 80 D. The modulus of the material used to construct the second section 126, on the other hand, can be generally greater than about 12 GPa, such as greater than about 15 GPa. For instance, in one embodiment, the modulus can range from about 12 GPa to about 25 GPa.

The second section 126 can be made from different thermoplastic polymers. In general, any suitable rigid thermoplastic polymer may be used to form the second section. Examples of polymers that may be used include, for instance, polyolefins including polyethylenes and polypropylenes, polystyrenes, polycarbonates, polyvinyl chlorides, and the like.

When forming the housing from a two-piece arrangement as shown in FIG. 7, in one embodiment, the first section 124 made from the softer material can be smaller in size than the second section 126. For instance, in one embodiment, the first section 124 can have a Z length that is minimized but still has the ability to engage the second section 126. For instance, the first section 124 can have a Z length that is less than about 20%, such as less than about 10%, such as less than about 5% of the total Z length of the signaling device. For instance, in one embodiment, the first section 124 can have a Z length that is from about 0.5% to about 5% of the total Z length of the signaling device.

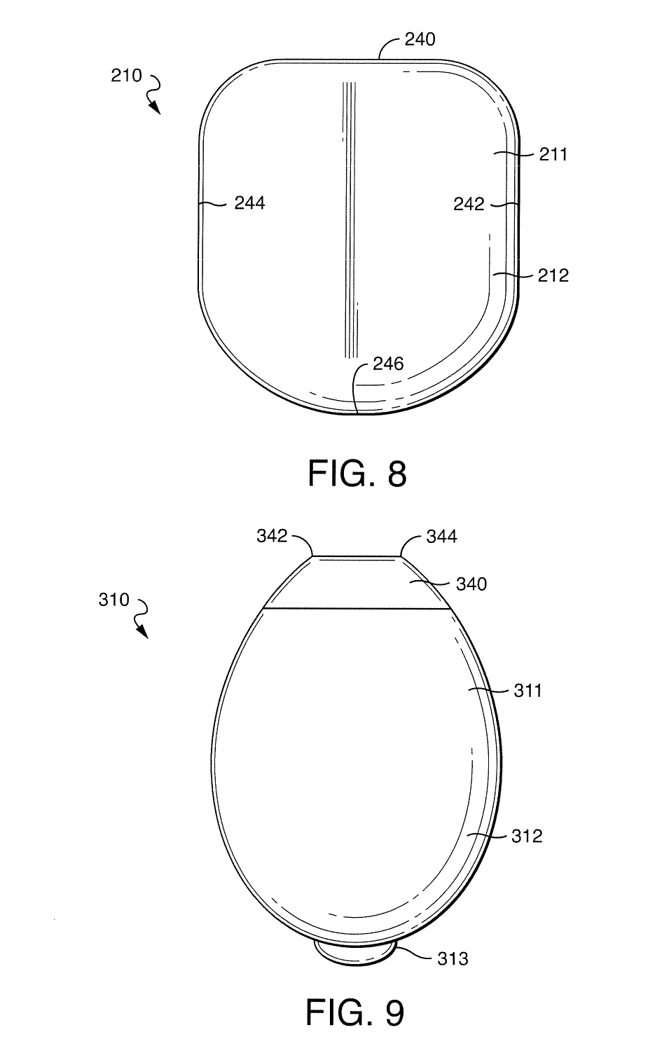

Referring now to FIGS. 8-10, various other embodiments of signaling devices made in accordance with the present disclosure are illustrated. Specifically, the signaling devices shown in FIGS. 8-10 illustrate other shapes of signaling devices made in accordance with the present disclosure. Although the overall shapes are different, however, it should be understood that the signaling device as illustrated in FIGS. 8-10 can generally have the same dimensions and can be made from the same materials as described with respect to the embodiment illustrated in FIGS. 6 and 7.

Referring to FIG. 8, for instance, a signaling device 210 is shown comprising a housing 211 containing internal components configured to generate a signal when activated. The signaling device 210 includes a first surface 212 that is designed to contact the body of a user, such as the back of a user when worn on a garment, such as an absorbent article. In this embodiment, the signaling device 210 includes three flat edges, 240, 242 and 244. The first side 212 of the signaling device 210 further includes an arcuate-shaped edge 246. As shown, edge 246 generally follows the same radius from the flat edge 242 to the flat edge 244.

Referring to FIG. 9, another embodiment of a signaling device generally 310 is shown. The signaling device 310 includes a housing 311 that defines a first surface 312 configured to lie adjacent to a person's body. The signaling device 310 also includes an attachment mechanism 313 for attaching the signaling device to a garment. For instance, the attachment mechanism 313 can comprise a clip that can be placed over a back waistband on an absorbent article so that the first surface 312 is positioned adjacent to a user's back.

In the embodiment illustrated in FIG. 9, the signaling device 310 generally has an oval-like shape. More particularly, the signaling device 310 includes a flat area 340 defining a first corner 342 spaced from a second corner 344. The first surface 312 extends from the two corners in an oval-like manner.

In one embodiment, the signaling device 310 can include the flat area 340 in order to form a hinge for the attachment mechanism 313.

The first surface 312 of the signaling device 310 can have an X-length and a Y length that are in the ranges described above with respect to the embodiment illustrated in FIGS. 6 and 7. For non-rectangular shapes, for instance, the X length and the Y length can be the maximum distance in the Y direction and the maximum distance in the X direction based upon the shape of the first surface 312.

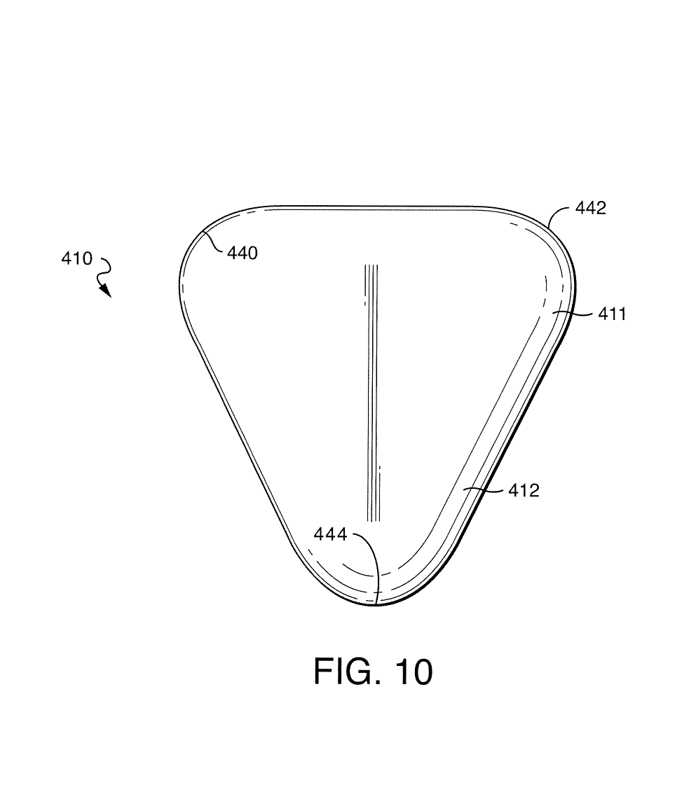

Referring to FIG. 10, still another embodiment of a signaling device 410 made in accordance with the present disclosure is shown. The signaling device 410 includes a housing 411 defining a first surface 412. In this embodiment, the first surface 412 has a triangular-like shape. In particular, the first surface 412 defines a first corner 440, a second corner 442, and a third corner 444. In accordance with the present disclosure, the triangular-like shape can generally be in the shape of an equilateral triangle or can have any other suitable triangular shape in which the sides have unequal lengths. In one particular embodiment, for instance, the signaling device 410 can have a shield-like shape in which a curved apex generally extends from a single flat edge.

Triangular-shaped signaling devices as shown in FIG. 10 may provide various advantages and benefits when used in accordance with the present disclosure. For instance, the present inventors have discovered that designs containing only three corners may serve to lower pressure values against the body when the signaling device is worn. In particular, triangular-shaped devices may have less sensitivity to the Z length or thickness of the device due to the shape.

In addition to the shapes illustrated in the figures, it should be understood that signaling devices can be made in accordance with the present disclosure having different and various other shapes. For instance, in one embodiment, a signaling device can be constructed having a compound shape.

The present disclosure may be better understood with reference to the following example.

### EXAMPLE

The following study was conducted in order to demonstrate some of the advantages and benefits of the present disclosure.

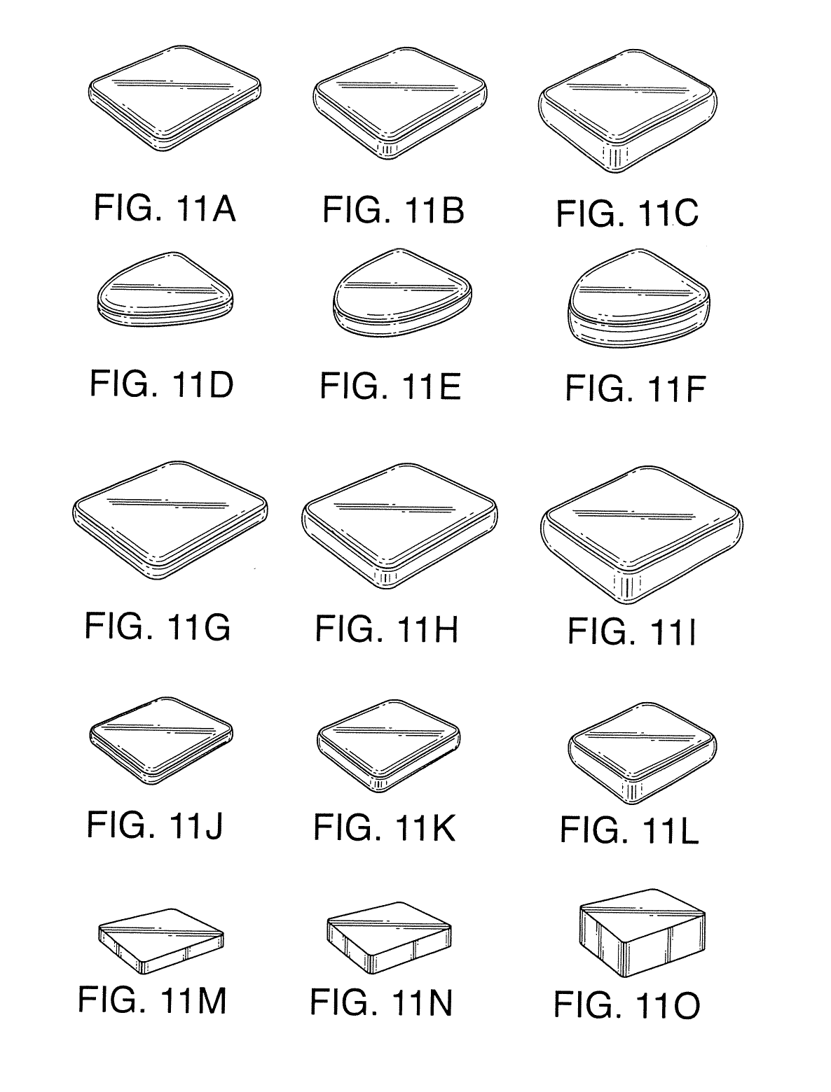

Referring to FIG. 11, fifteen different specimens (A-O) were tested according to this example. Each specimen was intended to represent the possible shape and dimensions of a signaling device. The specimens were constructed from LEXAN polycarbonate polymer available from General Electric.

Each of the specimens illustrated in FIG. 11 were attached to a disposable training pant along the back waistband of the pant. In this manner, the specimens were placed adjacent to the lower back of the wearer. In particular, the specimens were taped to the inside of the training pant along the waistline.

Small children were selected for the study. The children were weighed and measured the first day of the study. The children were then asked to wear disposable training pants containing one of the specimens illustrated in FIG. 11. After the training pant was secured to the wearer, each of the children was asked to sit and roll to their back onto a mat. The amount of pressure the specimens applied to the subject's body was measured.

Specifically, a pressure sensor grid was placed in between the specimen and the user's back. The pressure sensor used was Model No. 6077LLP sensor commercially available from Tekscan, Inc. of Boston, Mass. The sensor included software that recorded the output from a main sensor as discrete sensor areas. Each discrete sensor measures a pressure within the particular area. Using the software, a user can measure pressures over an area of interest, such as over the surface area of the signaling device, and get an average pressure for the particular area of interest.

Pressure measurements were taken during the entire time the child was asked to sit, lie down and sit back up. The pressure sensor was capable of providing an image of the peak pressures expressed over the entire area of the specimens during testing. These peak pressures were averaged over the surface area of the specimens to determine an average pressure. In addition, peak pressures were also recorded along the perimeter of each specimen. The peak perimeter pressures were averaged to find the average perimeter pressure for each specimen.

In FIG. 11, the shapes and sizes of the specimens are as follows. Specimens 11A, 11D, 11G, 11J, and 11M had a Z length or thickness of ¼ inch. Specimens 11B, 11E, 11H, 11K, and 11N had a Z length of ⅜ inches, while Specimens 11C, 11F, 11I, 11L, and 11O had a Z length of ½ inch.

Specimens 11A, 11B and 11C have an X and a Y length of 1.5 inches. Specimens 11G, 11H and 11I have an X length and a Y length of 1.75 inches. Specimens 11J, 11K, 11L, 11M, 11N, and 11O all have an X length and a Y length of 1.25 inches. Finally, Specimens 11D, 11E, and 11F have an X length of 1.25 inches and have a Y length of 1.25 inches. For Specimens 11D, 11E and 11F, the Y length is the longest length in the Y direction.

As shown in FIG. 11, specimens having rounded corners as described above were used in addition to specimens that did not include rounded corners.

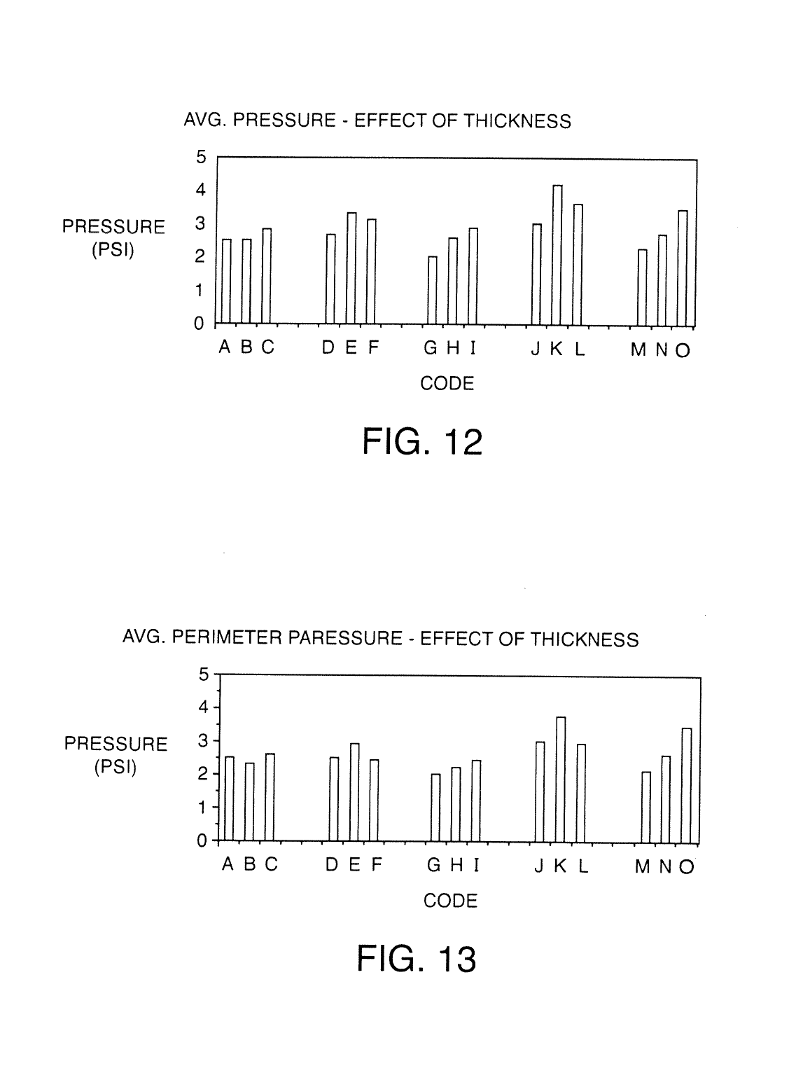

The results are illustrated in FIGS. 12 and 13.

As shown in the results, the amount of surface area was positively correlated with pressure values. More particularly, larger surface areas created lower average pressures. A similar trend was also found for average perimeter pressure.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed:

1. A signaling device configured to emit a signal when activated comprising:
an attachment mechanism for attaching the signaling device to a garment; and
a housing having a first side configured to contact a user when attached to a garment and a second and opposite side, the first side and the second side defining an X length and a Y length, the housing further having a thickness defining a Z length, the X length being from about 30 mm to about 80 mm, the Y length being from about 30 mm to about 80 mm, the Z length being less than about 15 mm, the first side defining at least two rounded corners, and wherein the radii of curvature at the two rounded corners between an edge in a Z direction and an edge in either an X direction or a Y direction is greater than about 1.5 mm⁻¹.

2. A signaling device as defined in claim 1, wherein the X length and the Y length are from about 30 mm to about 50 mm.

3. A signaling device as defined in claim 1, wherein the first side of the housing has a rectangular shape.

4. A signaling device as defined in claim 1, wherein the first side of the housing only includes two corners.

5. A signaling device as defined in claim 1, wherein the first side of the housing only includes three corners.

6. A signaling device as defined in claim 4, wherein the two corners of the first side define an end of the first side and wherein the first side has an oval-like shape extending from the two corners.

7. A signaling device as defined in claim 1, wherein the Z length of the housing is from about 5 mm to about 10 mm.

8. A signaling device as defined in claim 4, wherein the first side of the housing includes three generally flat sides and one arcuate-shaped side.

9. A signaling device as defined in claim 1, wherein the first side has a triangular shape.

10. A signaling device as defined in claim 7, wherein the X length and the Y length are from about 35 mm to about 55 mm.

11. A signaling device as defined in claim 1, wherein the housing is made from a material comprising a polymer.

12. A signaling device as defined in claim 1, wherein the first side of the housing is made from a material having a Shore A hardness of from about 25 A to about 70 A.

13. A signaling device as defined in claim 12, wherein the material used to form the first side of the housing has a modulus of less than about 10 GPa.

14. A signaling device as defined in claim 12, wherein the housing comprises a first section attached to a second section, the first section defining the first surface of the housing.

15. A signaling device as defined in claim 14, wherein the second section is made from a material having a Shore A hardness of from about 60 A to about 110 A and has a modulus of greater than about 12 GPa.

16. An absorbent article comprising an absorbent core positioned between a bodyside liner and an outer cover, the absorbent article defining a waist opening having a front and a back and two opposing leg openings when worn, the absorbent article further comprising a wetness sensing circuit, the wetness sensing circuit being configured to attach to the signaling device defined in claim 1 along the back of the waist opening so that the signaling device fits within a small of the back of a wearer.

17. An absorbent article as defined in claim 16, wherein the housing of the signaling device has a shape such that the signaling device produces an average pressure when worn on the absorbent article of less than about 3 psi.

18. An absorbent article as defined in claim 16, wherein the housing of the signaling device has a shape such that the signaling device produces an average perimeter pressure when worn on the absorbent article of less than about 3 psi.

19. A signaling device configured to emit a signal when activated comprising:
an attachment mechanism for attaching the signaling device to a garment; and
a housing having a first side configured to contact a user when attached to a garment and a second and opposite side, the first side and the second side defining an X length and a Y length, the housing further having a thickness defining a Z length, the X length being from about 30 mm to about 80 mm, the Z length being less than about 15 mm, the first side of the housing being made from a material that has a Shore A hardness of from about 25 A to about 75 A and has a modulus of less than about 10 GPa.

20. A signaling device as defined in claim 19, wherein the housing comprises a first section attached to a second section, the first section defining the first surface of the housing.

21. A signaling device as defined in claim 20, wherein the second section is made from a material having a Shore A hardness of from about 60 A to about 110 A and has a modulus of greater than about 12 GPa.

22. A signaling device as defined in claim 19, wherein the first side of the housing has a rectangular shape.

23. A signaling device as defined in claim 19, wherein the first side of the housing only includes two corners.

24. A signaling device as defined in claim 19, wherein the first side of the housing only includes three corners.

25. An absorbent article comprising an absorbent core positioned between a bodyside liner and an outer cover, the absorbent article defining a waist opening having a front and a back and two opposing leg openings when worn, the absorbent article further comprising a wetness sensing circuit, the wetness sensing circuit being configured to attach to the signaling device defined in claim 20 along the back of the waist opening so that the signaling device fits within a small of the back of a wearer.
